(19) United States
(12) Patent Application Publication
(10) Pub. No.: US 2011/0200497 A1
(43) Pub. Date: Aug. 18, 2011

(54) APPARATUS FOR TREATMENT OF TISSUE SPECIMENS

(75) Inventors: Torstein Ljungmann, Nesoddtangen (NO); Oystein Ljungmann, Siggerud (NO)

(73) Assignee: Dako Instrumec AS, Oslo (NO)

(21) Appl. No.: 13/095,010

(22) Filed: Apr. 27, 2011

Related U.S. Application Data
(63) Continuation of application No. 10/582,561, filed on Apr. 6, 2007.

(51) Int. Cl. A61L 2/04

(52) U.S. Cl. 422/307

(57) ABSTRACT

An apparatus for heat treatment of tissue specimens, comprising a pressure cooker (1) for cooking of tissue specimens, a temperature sensor (5) and a pressure sensor (6) connected to the pressure cooker, and a control unit (15) for time-controlled heat treatment of the tissue specimens in the pressure cooker (1). The control unit is arranged to control a programmed step-by-step heating course, with a programmed time duration on each temperature step, from a chosen start temperature up to a chosen maximum temperature. The apparatus preferably comprises a vent valve (8) coupled to the pressure cooker, and the control unit (15) then is also arranged to control a programmed step-by-step cooling course, from the chosen maximum temperature down to a chosen final temperature.

This application is a continuation of U.S. patent application Ser. No. 10/582,561 filed Apr. 6, 2007 entitled “Apparatus for Treatment of Tissue Specimens” which is a 371 filing of PCT/NO2003/000415 filed Dec. 12, 2003, the disclosures of which incorporated herein by reference.

The invention relates to an apparatus for heat treatment of tissue specimens, comprising a pressure cooker for cooking of the tissue specimens, a temperature sensor and a pressure sensor connected to the pressure cooker, and a control unit for time-controlled heat treatment of the tissue specimens in the pressure cooker.

In heat treatment of tissue specimens in connection with so-called immuno preparation it has been common practice to treat the tissue specimens in an ordinary cooking process for about 1 hour before the specimens are transferred to an immuno stainer. Within the prior art there is an immuno stainer that heats the specimens to about 95° C. before the very immuno staining process. Today, this is considered to be sufficient among recognized researchers.

Another known alternative is to use a microwave oven which can also heat the liquid and the tissue specimens to above the boiling point.

There have also been used pressure cookers of plastics in microwave ovens in order to get above the boiling point. The use of a microwave oven may, however, entail hot/cold spots which may entail internal temperature differences in each tissue specimen, and also a difference from specimen to specimen in one and the same process. In addition there will also be variations from cycle to cycle. This technology therefore has big problems with the reproducibility.

Experiments/research have shown, however, that it is of great importance to the quality of the immuno staining that the tissue specimens obtain a higher temperature than 100° C. By means of a higher temperature also the time of the heat treatment will be reduced. This occurs only at temperatures around 115° C. The result is that the tissue specimens get a sharper and smoother staining.

A device for heating of biological specimens, and which comprises a pressure cooker, is known from U.S. Pat. No. 6,580,056. This patent shows a pressure cooker having a heating element integrated therein, a pressure gauge and a temperature gauge controlled by a controller seeing to it that a chosen temperature is obtained at a given pressure in a given time. When the set time is reached, an alarm will give a warning.

A drawback of the above-mentioned prior art is that the users can not use different programs for different temperature courses of the heating process, according to the type tissue specimens and the subsequent staining process. Further, the cooling process is not controllable, as it is only based on air cooling after the heating element is disconnected. This means that the cooling time is dependent on the liquid quantity in the pressure cooker and the quantity of tissue specimens placed in the liquid. The cooling time with a large liquid volume in the pressure cooker will then be long, but the time will be correspondingly shorter with little liquid and only a few specimens in the pressure cooker. Therefore, this does not give the user a sufficient control over the total process, and the result is that the desirable reproducibility is not obtained.

Thus, it is a main object of the invention to provide an apparatus enabling programming of a step-by-step heating course, i.e. a desired time at a given temperature in steps up to a desired maximum temperature in a given time.

An additional object of the invention is to provide such an apparatus which also enables programming of a step-by-step cooling course, i.e. a desired time at a given temperature in steps down to a desired minimum temperature.

For achieving the above-mentioned objects there is provided an apparatus of the introductorily stated type which, according to the invention, is characterised in that the control unit is arranged to control a programmed step-by-step heating course, with a programmed time duration on each temperature step, from a chosen start temperature up to a chosen maximum temperature.

An apparatus with which also the above-stated additional object is achieved, is characterised in that it comprises a vent valve which is coupled to the pressure cooker, and that the control unit is also arranged to control a programmed step-by-step cooling course, from the chosen maximum temperature down to a chosen final temperature. With this embodiment one can, by regulating the steam pressure in the pressure cooker, see to it that the programmed temperatures are not exceeded, even with an efficient heating up to the programmed temperatures, without the result becoming a superheating. The temperature will be able to be controlled in a very quick and stable manner up to the programmed temperature levels. Correspondingly, by reducing the steam pressure, one can quickly lower the temperature down to about 100° C.

The control unit of the apparatus preferably comprises a processor unit which controls the relevant temperature courses by means of a data program.

It is also advantageous that the apparatus comprises a vacuum pump which is coupled to the pressure cooker via an electric valve, for reducing the pressure in the pressure cooker to a desired value. With this embodiment one can lower the pressure in the pressure cooker additionally, and in this manner reduce the temperature in a controlled and quick manner down to for example 70° C. (the boiling point at 0.5 atm.). One can also start a heating process with an underpressure, so that one gets a controlled vaporization and temperature control below 100° C., for example at 90 or 95° C.

In a practical embodiment of the apparatus according to the invention, the pressure cooker and the control unit are integrated in a treatment apparatus which is also arranged to carry out dewaxing (i.e. removal of paraffin) of tissue specimens on microscope slides before the heat treatment in the pressure cooker.

By means of the apparatus according to the invention, the user is unable to program and run any chosen temperature cycle, both up and down, step by step. The user can for himself program time phases with different temperatures, and also program a maximum temperature (which must be below the maximum temperature at which the pressure relief valve opens) for a programmed time. This gives the user the advantage that the whole process is controlled and repeatable, and that hot steam from the pressure cooker is reduced. Further, the program will be able to warn when a suitable handling temperature of the tissue specimens in the pressure cooker is attained.

The invention will be further described below in connection with an exemplary embodiment with reference to the drawings, wherein

FIG. 1 shows a schematic view of a pressure cooker to which there are connected pressure and temperature sensors, electric control valves and a vacuum pump;

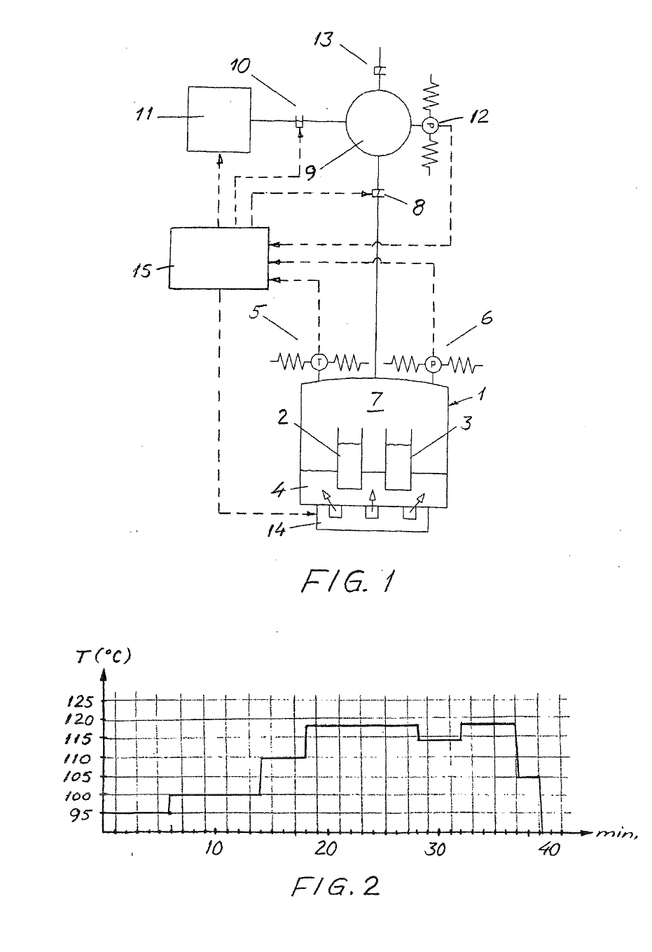

FIG. 2 is a diagram showing an example of a program protocol with process verification;

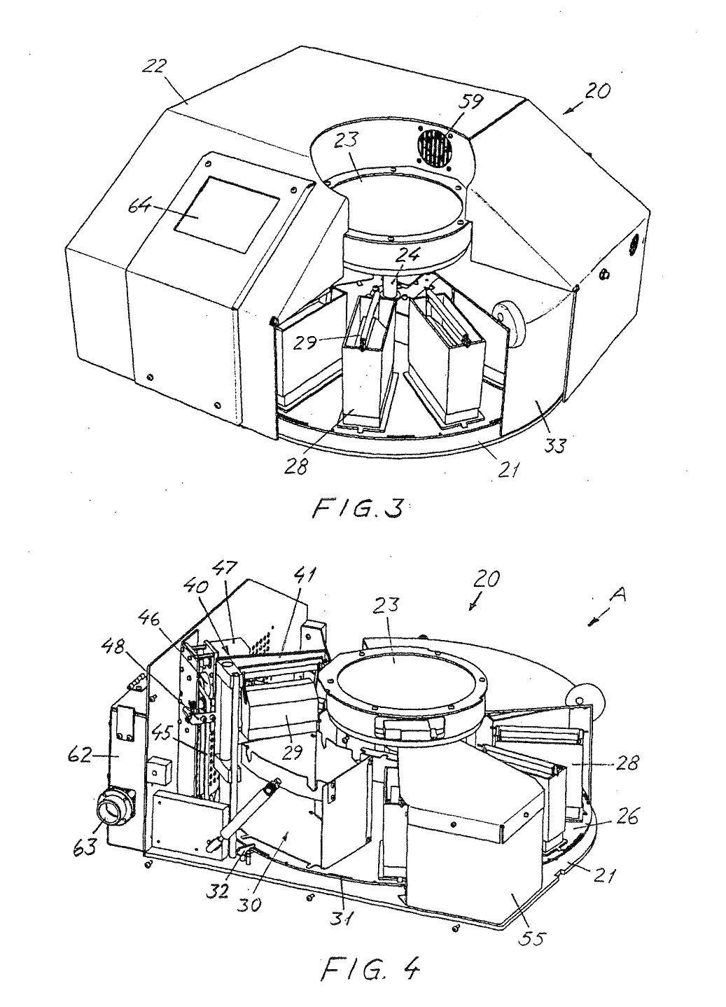

FIG. 3 shows a perspective view of an apparatus which is provided with the necessary means for operation of the pressure cooker, and which is also arranged for dewaxing of tissue specimens on microscope slides;

FIG. 4 shows a perspective view of the apparatus in FIG. 3, as viewed in the direction from the left in FIG. 3, and wherein a bonnet over the apparatus has been removed;

FIG. 5 shows the apparatus according to FIG. 4, as viewed in the direction of the arrow A in FIG. 4;

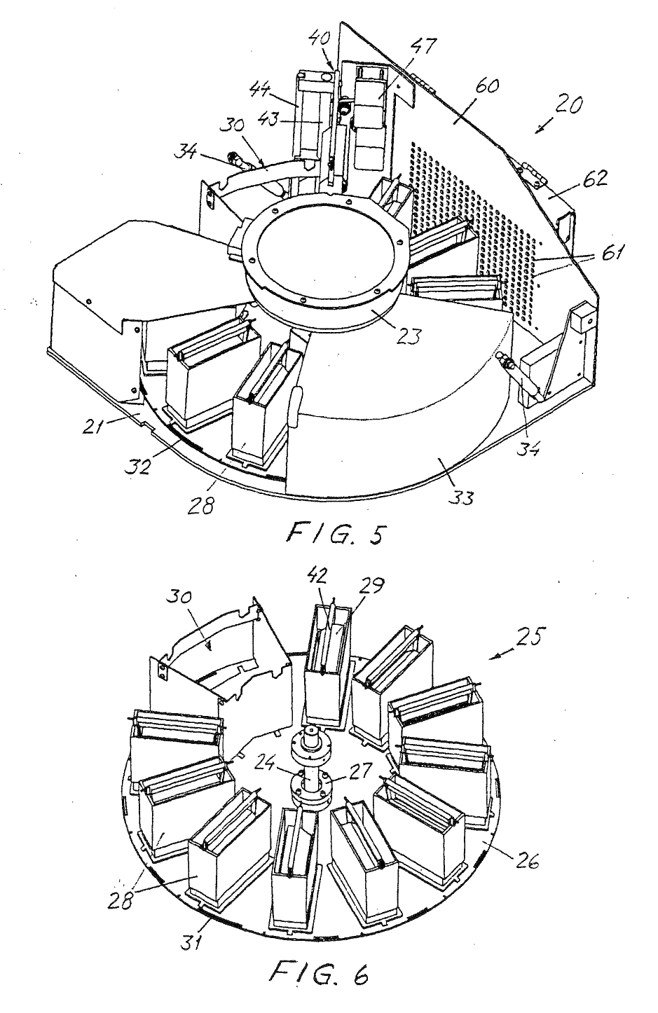

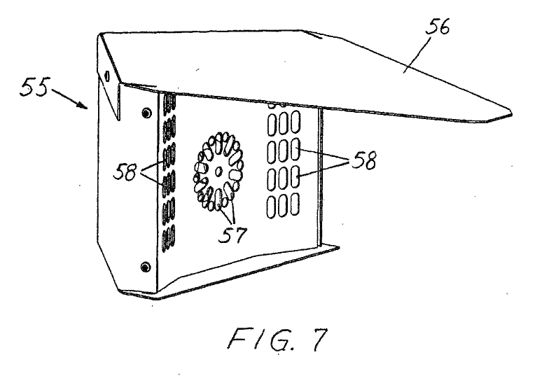

FIG. 6 shows a perspective view of a revolving unit forming part of the apparatus according to FIGS. 3-5; and

FIG. 7 shows a perspective view of a heating stove forming part of the apparatus according to FIGS. 3-5.

That part of the apparatus according to the invention directly relating to heat treatment of tissue specimens placed in a pressure cooker, will be described with reference to the schematic view in FIG. 1.

In FIG. 1 there is shown a pressure cooker 1 in which there are placed two vessels 2 and 3 of which each contains a “buffer” liquid (a salt liquid mixture), and which are placed in a suitable water quantity 4 at the bottom of the pressure cooker. The vessels 2 and 3 are intended to receive baskets with microscope slides, wherein tissue specimens which have been dewaxed in a preceding dewaxing process, are placed on the microscope slides. Such a dewaxing process here is presupposed to be carried out in the treatment apparatus shown in FIGS. 3-5, and which will be described later.

The pressure cooker 1 in FIG. 1 is provided with a temperature sensor 5 and a pressure sensor 6 for sensing of the temperature and pressure, respectively, in the inner space 7 of the pressure cooker. The inner space 7 is connected to an electric valve 8 constituting a first vent valve which may be opened during operation to lower the steam pressure for a quick and controlled cooling of the pressure cooker. The cooker is connected via the valve 8 to a container 9 which is connected to a vacuum pump 11 via an additional electric valve 10. The container 9 is connected to a pressure sensor 12, and further to a third electric valve 13 which, when opened, connects the container space to the atmosphere, and thus constitutes a pressure equalizing valve.

For heating of the pressure cooker there is arranged a heating element 14 which in the illustrated embodiment consists of a usual hot plate.

As shown in FIG. 1, there is arranged a control unit 15 for controlling the heat treatment of the tissue specimens in the pressure cooker. As symbolically suggested with stippled lines, the control unit receives signals from the different sensors, and based on these signals the control unit is arranged to control the different valves, the vacuum pump and the current supply to the hot plate, to control the relevant temperature cycle for the heat treatment of the tissue specimens.

In order to be able to carry out the topical operations in the course of a treatment cycle, the control unit 15 comprises a processor or central processing unit (CPU) that controls the relevant temperature courses by means of a data program. For this purpose there is provided a specially developed software comprising a number of program for controlling respective ones of the desired treatment cycles. Thus, by means of the topical/relevant program the control unit can control a programmed step-by-step heating course, with a programmed time duration on each temperature step, from a chosen start temperature up to a chosen maximum temperature. As mentioned above, the pressure cooker is provided with a vent valve 8 which, when opened, lowers the steam pressure for quick and controlled cooling. This arrangement enables that the control unit can also be arranged to control a programmed step-by-step cooling course, from the chosen maximum temperature down to a chosen final temperature.

By means of the vacuum pump 11 which is connected to the pressure cooker, the pressure in the cooker may be lowered additionally, and thereby the temperature can be reduced in a quick and controlled manner to e.g. 70° C. (boiling point at 0.5 atm.).

FIG. 2 is a diagram showing an example of a program protocol with process verification. The diagram shows the temperature in the pressure cooker as a function of time during a chosen treatment cycle. As appears, the topical temperature cycles comprise eight phases, wherein the temperature and time duration for the individual phases are as follows:

| | Temp. (° C.) | Time periode (min.) |
|---|---|---|
| Phase 1 | 95 | 6 |
| Phase 2 | 100 | 8 |
| Phase 3 | 110 | 4 |
| Phase 4 | 118 | 10 |
| Phase 5 | 114 | 4 |
| Phase 6 | 118 | 5 |
| Phase 7 | 105 | 2 |
| Phase 8 | 90 | 2 |

As mentioned above, the pressure cooker and the control unit with the associated components in the embodiment shown in the drawing are integrated in a treatment apparatus which is also arranged to carry out dewaxing of tissue specimens on microscope slides before the heat treatment in the pressure cooker. This treatment apparatus will be described with reference to FIGS. 3-7.

In FIGS. 3-5 the treatment apparatus in its entirety is designated by the reference numeral 20. The different parts and elements forming part of the apparatus are mounted on a chassis which, inter alia, comprises a base plate 21, and which is generally covered by a bonnet 22.

As shown, the apparatus comprises a hot plate 23 for heating of the pressure cooker 1 with the tissue specimens placed therein. The hot plate is carried by a column 24 supported by the base plate 21. Under the hot plate there is mounted a revolving unit 25 (see FIG. 6) comprising a circular support plate 26 which is mounted for rotation about the column 24 by means of a central bearing 27. The plate 26 supports an annular arrangement of vessels 28 for the receipt of respective baskets 29 in which there are placed microscope slides (not shown) with tissue specimens which are to be treated in liquid baths in the vessels. The liquid baths consist of suitable reagents for dewaxing and treatment of the tissue specimens. In the illustrated embodiment, ten such vessels 28 are arranged on the support plate 26. Further, a loading magazine 30 for vessels to be retreated in the apparatus is placed on the plate. In the illustrated embodiment the loading magazine comprises two stations (station 1 and 2), so that it can receive two baskets at the time.

The revolving unit 25 can be rotated in steps by a driving motor (not shown) in the form of a stepping motor having a drive wheel which may be in engagement with the support plate 26 on the underside thereof. The plate may also be supported by additional supporting wheels on the underside of the plate.

The support plate is provided along its circumference with a number of circumferentially extending gaps or slots 31 which are located between the vessels 28 and cooperate with a so-called reading fork 32 for controlling the stepwise movement of the support plate by means of the mentioned driving motor. One of the slots is arranged in the region of the loading station 30 and is wider than the other slots to mark a “home position” for the revolving unit 25.

As best shown in FIG. 5, there is arranged a rotatable sliding door 33 which is rotatably mounted on the column 24, and thereby can be turned so that it covers or uncovers a desired number of the vessels 29. When the sliding door is slid to the closed position, it actuates a sensor (not shown) causing an automatic start of an operation cycle, as described later.

Further, there are shown a pair of gas springs 34 connected between the chassis and the bonnet 22 of the apparatus, in order to raise the bonnet to provide access to the loading magazine 30 and the vessels 28.

A appears from FIGS. 4 and 5, a hoist or lifting device 40 is arranged next to the revolving unit 25, for lifting and lowering of baskets 29 that are to be transferred from the loading magazine to the individual vessels 28, or from one vessel to another. The hoist device is also shown to comprise a boom 41 which is adapted for engagement with a suspension rod 42 on the individual baskets, and which is fastened to a bracket 43 which is slidably mounted on a vertical support column 44. The bracket 43 is coupled to a driving belt 45 running over upper and lower guide wheels 46 (only an upper wheel is shown), where the upper wheel is driven by a hoist motor 47. The movement of the hoist is controlled by means of a lifting fork 48 detecting the position of the boom 41.

The apparatus is provided with a heating stove 55 for heating and softening of the paraffin in which the tissue specimens on the microscope slides are embedded. The heating stove is shown in FIG. 7 and comprises a hot plate 56, a number of openings 57 for blowoff of hot air, and a number of openings 58 for return air.

As appears from FIG. 3, the apparatus further is provided with a blower 59 mounted on the chassis next to the hot plate 23 and having the function to blow surrounding air towards the pressure cooker when this is to be cooled. Further, a rear wall 60 of the chassis of the apparatus is provided with a perforated portion having openings 61 forming a ventilation outlet for discharge of gases and fumes from the reagent vessels 28 in the apparatus. These gases/fumes flow into a box 62 containing a cleaning carbon filter (not shown), and which is provided with a ventilation outlet 63.

For operation of the apparatus and control of the relevant operation cycles, the apparatus is provided with a display having a touch screen 64 as shown in FIG. 3. The operation points of the touch screen are interconnected with the aforementioned control unit 15, in such a manner that this can be actuated via the touch screen and thereby control the relevant desired operation cycle in accordance with the chosen program.

The manner of the operation of the apparatus during an operation cycle will be described below.

By touching the touch screen 64, the revolving unit 25 turns so that station 1 in the loading magazine 30 is exposed from the chassis, so that a first basket 29 (“basket 1”) can be placed on this station. Thereafter one may either touch “Start” for a chosen program and obtain autostart when the rotatable sliding door 33 is closed, or one may choose “basket 2”, which entails that the revolving unit is turned and exposes station 2. When the sliding door is closed, the revolving unit 25 with the loading magazine is turned ahead to the heating stove 55 which is then connected, and the paraffin is softened at a programmed temperature for a programmed time.

Thereafter the loading magazine 30 on the revolving unit is turned ahead to the hoist device 40 which by turns lifts the baskets 1 and 2 into their respective baths in the vessels 28, and further to the remaining programmed baths in the treatment process.

When the dewaxing process is finished, the basket/baskets is/are lifted manually into one or both of the two vessels placed in the pressure cooker. The lid of the pressure cooker is put on, and a programmed heating cycle is started. The two baths in the vessels contain the same or different types of “buffers”, and are placed in approximately half a liter of usual water at the bottom of the pressure cooker. Alternatively, the cooker itself may be filled with a buffer, so that one obtains space for 5×30 tissue specimens.

After finished cooking (maximum temperature ca. 120° C.), the heating stove 55 is switched off and the cooling blower 59 is switched on and draws room-tempered air around the pressure cooker. Further, the vent valve 8 is opened to lower the steam pressure for rapid and controlled cooling. After having reached 100° C., the vacuum pump 11 is started, which pump further reduces the pressure in the pressure cooker, so that the temperature in a rapid and controlled manner is reduced quite down towards 70° C.

What is claimed is:

1. An apparatus for heat treatment of tissue specimens, comprising a pressure cooker (1) for cooking of the tissue specimens, a temperature sensor (5) and a pressure sensor (6) connected to the pressure cooker, and a control unit (15) for time-controlled heat treatment of the tissue specimens in the pressure cooker (1), the control unit (15) being arranged to control a programmed step-by-step heating course, with a programmed time duration on each temperature step, from a chosen start temperature up to a chosen maximum temperature, wherein it comprises a vent valve (8) coupled to the pressure cooker (1), and that the control unit (15) also is arranged to control a programmed step-by-step cooling course by controlling pressure, from the chosen maximum temperature down to a chosen final temperature, wherein both the programmed step-by-step heating course and the programmed step-by-step cooling course are controlled by the control unit via a data program.

2. An apparatus according to claim 1, wherein the control unit (15) comprises a processor unit which controls the relevant temperature courses by means of a data program.

3. An apparatus according to claim 1, wherein it comprises a vacuum pump (11) which is connected to the pressure cooker (1) via an electric valve (10), to reduce the pressure in the pressure cooker to a desired value.

4. An apparatus according to claim 1, wherein the pressure cooker (1) and the control unit (15) are integrated in a treatment apparatus (20), the treatment apparatus operable to perform dewaxing of tissue specimens on microscope slides before the heat treatment in the pressure cooker.

5. An apparatus according to claim 4, wherein the treatment apparatus (20) comprises a hot plate (23) for heating of the pressure cooker (1) with the tissue specimens, a revolving unit (25) arranged under the hot plate and comprising a rotatable plate (26) supporting an annular arrangement of vessels (28) for receiving baskets (29) with microscope slides and a loading magazine (30) for baskets, a driving motor for controlled step-by-step rotation of the revolving unit (25), and a hoist device (40) for lifting and lowering of the baskets which are to be transferred from the loading magazine (30) to the individual vessels (28) and from vessel to vessel.

6. An apparatus according to claim 5, wherein it includes a heating stove (55) for heating of baskets (29) in the loading magazine (30).

7. An apparatus according to claim 2, wherein it comprises a vacuum pump (11) which is connected to the pressure cooker (1) via an electric valve (10), to reduce the pressure in the pressure cooker to a desired value.

8. An apparatus according to claim 2, wherein the pressure cooker (1) and the control unit (15) are integrated in a treatment apparatus (20), the treatment apparatus operable to perform dewaxing of tissue specimens on microscope slides before the heat treatment in the pressure cooker.

9. An apparatus according to claim 3, wherein the pressure cooker (1) and the control unit (15) are integrated in a treatment apparatus (20), the treatment apparatus operable to perform dewaxing of tissue specimens on microscope slides before the heat treatment in the pressure cooker.
